(19) United States
(12) Patent Application Publication
(10) Pub. No.: US 2026/0174972 A1
(43) Pub. Date: Jun. 25, 2026

(54) Non-Round Reservoir Injection Pen with Telescoping Screws

(71) Applicant: Embecta Corp., Parsippany, NJ (US)

(72) Inventors: Alessandro E. Pizzochero, Chelmsford, MA (US); Dana Cote, Kensington, NH (US); Mark Wood, Sterling, MA (US)

(73) Assignee: Embecta Corp., Parsippany, NJ (US)

(21) Appl. No.: 19/255,349

(22) Filed: Jun. 30, 2025

Related U.S. Application Data
(63) Continuation of application No. PCT/US2024/011402, filed on Jan. 12, 2024.
(60) Provisional application No. 63/479,772, filed on Jan. 13, 2023.
(60) Provisional application No. 63/507,657, filed on Jun. 12, 2023.

(51) Int. Cl. A61M 5/315; A61B 17/70; A61B 17/86; A61B 17/88; A61M 5/24; A61M 5/32

(52) U.S. Cl. CPC A61M 5/31593; A61B 17/7032; A61B 17/7035; A61B 17/8605; A61B 17/8875; A61M 5/24; A61M 5/31541; A61M 5/31551; A61M 5/3157; A61M 5/3202

(57) ABSTRACT

A delivery pen includes a reservoir including a non-circular shape, a plunger positioned in the reservoir, and a screw mechanism at least partially inserted within the reservoir. The screw mechanism includes a drive shaft including one or more protruding member at a proximal end and an elongated member extending longitudinally from the proximal end, an inner screw concentrically engaging the elongated member, an outer housing including a circular section rotatably engaging the one or more protruding member and a non-circular section sized to fit within the reservoir, and a pusher disposed between the inner screw and the outer housing to linearly translate the plunger so as to dispense a medication from the reservoir.

### CROSS-REFERENCE TO RELATED APPLICATIONS

This application is a bypass continuation patent application of PCT International Patent Application No. PCT/US2024/011402, filed Jan. 12, 2024, which claims the benefit of and priority to U.S. Provisional Patent Application No. 63/479,772, filed Jan. 13, 2023, and U.S. Provisional Patent Application No. 63/507,657, filed Jun. 12, 2023, the contents of each of which are incorporated herein by reference in their entirety.

### FIELD

This disclosure relates to non-circular medicament reservoirs and associated screw mechanisms in delivery pens for delivery of therapeutic compounds.

### BACKGROUND

For treatment of some diseases and conditions, it is often desirable to inject medication directly into the tissue of a patient. For instance, pen injection or pump devices are used to inject medicaments into tissue areas. Currently, pen injection or pump devices are fairly bulky, with limited options to achieve medicament administration into a tissue that are often complex or uncomfortable to use. Therefore, there is a need for medicament delivery systems that are easier to use and ergonomic to fit different lifestyles.

### SUMMARY

The above and other problems are overcome by embodiments of the present disclosure.

The present disclosure relates to a delivery pen including: a reservoir including a non-circular shape; a plunger positioned in the reservoir; and a screw mechanism at least partially inserted within the reservoir, the screw mechanism including: a drive shaft including one or more protruding member at a proximal end and an elongated member extending longitudinally from the proximal end; an inner screw concentrically engaging the elongated member; an outer housing including a circular section rotatably engaging the one or more protruding member and a non-circular section sized to fit within the reservoir; and a pusher disposed between the inner screw and the outer housing to linearly translate the plunger so as to dispense a medication from the reservoir.

The present disclosure relates to a delivery pen including: a reservoir including a non-circular shape; a plunger positioned in the reservoir; and a screw mechanism at least partially inserted within the reservoir, the screw mechanism including: a drive shaft including one or more protruding member at a proximal end and an elongated member extending longitudinally from the proximal end; an inner screw concentrically engaging the elongated member, the inner screw including a wide section and a narrow section extending longitudinally from the wide section; an outer screw concentrically engaging the wide section, the outer screw including a circular section rotatably engaging the one or more protruding member, and a non-circular section sized to fit within the reservoir; and a pusher disposed between the narrow section and the outer screw to linearly translate a plunger to dispense a medication from the reservoir.

The present disclosure relates to a delivery pen including: a main body; a cap engaged to the main body; a torque coupling component including inner axial slots, wherein the torque coupling component is housed with the main body; a screw mechanism including: a drive shaft including a proximal end and an elongated member extending distally from the proximal end, wherein the proximal end of the drive shaft includes one or more protruding member configured to mate with the inner axial slots of the torque coupling component; an inner screw concentrically engaging the elongated member and including a first outer threading; an outer housing including a circular section and a non-circular section, wherein a proximal end of the circular section includes an indent configured to engage the one or more protruding member such that the drive shaft can rotate about but cannot longitudinally extend from the indent; and a pusher, wherein an inner surface of the pusher includes inner threading configured to mate with the first outer threading of the inner screw; a reservoir housed within the cap, the reservoir including a non-circular shape, an outlet port at a distal end, and a plunger movable along a longitudinal axis of the reservoir, the plunger configured to engage a distal end of the pusher and to provide a seal with respect to inner walls of the reservoir to prevent fluid provided in a fluid chamber defined on a first side of the plunger from leaking into a portion of the reservoir defined by a second side of the plunger; and a dose knob engaged to the torque coupling component, wherein the dose knob is rotatable with respect to the main body for adjusting a volume of fluid delivery such that rotation of the dose knob in a first direction facilitates axial translation of the drive shaft away from a proximal end of the main body, and rotation of the dose knob in a second direction facilitates axial translation of the drive shaft toward the proximal end of the main body.

The present disclosure relates to a delivery pen including: a non-circular reservoir; and a screw mechanism at least partially inserted within the non-circular reservoir, the screw mechanism including: a drive shaft, wherein a proximal end of the drive shaft includes one or more protruding member; an inner screw including a wide section and narrow section, wherein an inner surface of the inner screw is keyed to engage a distal end of the drive shaft such that torque applied to the drive shaft is transferred to the inner screw, the wide section includes a first outer threading, and the narrow section includes a second outer threading; an outer screw including a circular section and a non-circular section, wherein the circular section includes inner threads configured to engage with the first outer threading, and wherein a proximal end of the circular section includes an indent configured to engage the one or more protruding member such that the drive shaft can rotate about but cannot longitudinally extend from the indent; and a pusher distally configured to engage a plunger, wherein an inner surface of the pusher includes inner threading configured to mate with the second outer threading of the inner screw.

Example embodiments of the present disclosure prevents rotation of a plunger in circular fluid delivery devices, while retaining the features of highly reliable and proven systems such as medication pens and pen needles, syringes, or more expensive, non-portable pumping systems that employ a lead screw drive mechanism.

### BRIEF DESCRIPTION OF THE DRAWINGS

The features of the disclosure are set forth with particularity in the appended claims. The patent or application file contains at least one drawing executed in color. Copies of this patent or patent application publication with color drawing(s) will be provided by the Office upon request and payment of the necessary fee. A better understanding of the features of the present disclosure will be obtained by reference to the following detailed description that sets forth illustrative embodiments, in which the principles of the disclosure are utilized, and the accompanying drawings of which:

FIGS. 1A-1D illustrate perspective view of the delivery pen, according to aspects of this disclosure. FIGS. 1A-1D illustrate perspective views of the delivery pen with a cap, without the cap and with a needle installed, without the cap and without the needle, and a cross-sectional view along a longitudinal axis of the delivery pen, respectively;

FIGS. 2A-2B illustrate cross-sectional views of a screw mechanism in nested and extended positions, respectively, according to some aspects of this disclosure; FIGS. 2C-2D are isometric views of a screw mechanism illustrating a screw mechanism in nested position, according to some aspects of this disclosure;

FIG. 3 illustrates an isometric cross-sectional view of the screw mechanism in a partially extended position, according to some aspects of this disclosure;

FIG. 4 illustrates stop features of a drive shaft and inner screw, according to some aspects of this disclosure;

FIG. 5 illustrates stop features of an inner screw and plunger, according to some aspects of this disclosure;

FIG. 6 is a cross-sectional view illustrating a screw mechanism in a partially extended position, according to some aspects of this disclosure;

FIG. 7 is a cross-sectional view illustrating a screw mechanism in a partially extended position, according to some aspects of this disclosure;

FIG. 8 is a cross-sectional view illustrating an alternative stop features of a drive shaft and inner screw, according to some aspects of this disclosure;

FIGS. 9A-9B illustrate transparent views of a screw mechanism in nested and extended positions, respectively, according to some aspects of this disclosure;

FIG. 10 illustrates a perspective transparent view of the screw mechanism in a partially extended position, according to some aspects of this disclosure;

FIG. 11 illustrates a front view of a delivery pen, according to some aspects of this disclosure;

FIGS. 12A-12B illustrate perspective views of a torque coupling component alone and assembled with the screw mechanism, respectively, according to some aspects of this disclosure;

FIG. 13 illustrates a perspective view of the delivery pen with a storage compartment in an arrow shape, according to some aspects of this disclosure; and

FIG. 14 illustrates a perspective view of the delivery pen with a storage compartment in an extended shape, according to some aspects of this disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides a screw mechanism for enabling a fluid or medicament delivery device, such as a delivery pen, to work with a reservoir that has a non-circular cross-section in fluid delivery devices. In some embodiments, implementing non-circular cross-section reservoirs allow for delivery pens to be shorter, more ergonomic and provide more storage space. Because many pen injection devices have multiple parts and limited medicament capacity, there is a need for medicament delivery systems that increase functionality for users while reducing size and form factor, where the capital equipment used to determine the dose amount administered to a patient is modular from the pen injection or pump device. A pump with a reduced size of the basic mechanism enables the potential to use a larger reservoir while minimizing size. This is a potential advantage for patients as they can stretch the use of a single pen for more days. Telescoping screw mechanisms may be used for advancement of a plunger within a fluid reservoir for controlled dispensing of fluids. In some instances, for example a medical fluid dispensing device having a round reservoir, anti-rotation features are required to enable movement of such telescoping screw mechanisms between a nested configuration and an extended configuration, otherwise the plunger is prevented from advancing through the reservoir. Non-circular reservoirs, however, may avoid this issue by preventing rotation of the screw mechanism as doses are administered, thus allowing for extension of the screw mechanism through the reservoir without additional anti-rotation features. The assembly described herein overcomes this challenge and provides anti-rotation capability to the telescoping screw mechanism for non-circular reservoirs.

Illustrative embodiments in this disclosure relate generally to telescoping screw mechanisms for use in fluid delivery devices such as delivery pens. Illustrative embodiments relate generally to nesting telescopic screws with a non-circular section for controllably extending or retracting a plunger in a syringe non-circular reservoir that do not affect reservoir volume to ensure biocompatibility, that are fully retractable outside reservoir, and engage with the reservoir for anti-rotation control. Telescoping screw-driven mechanisms have been designed in pens for dispensing medication but rely on restraining rotation in one of the screws in order to generate extension, otherwise the system would simply spin without advancing. This is required as the opposite end of the telescoping screw is fixed with the body of the device and provides a reference for the rotation and advance of one of the screws. In an embodiment, a non-circular shape of the reservoir can be used to create anti-rotation and thus to enable extension of a plunger from a nested position. This disclosure relates to anti-rotation mechanisms with a substantially reduced length.

Implementing a non-circular reservoir for anti-rotation can eliminate or reduce the need for additional anti-rotation mechanisms in the fluid delivery device. In some embodiments, by reducing or eliminating these additional anti-rotation mechanisms, increased space can be available within the fluid delivery device for other components, such as a dose measurement encoder. In some embodiments, by reducing or eliminating the additional ant-rotation mechanisms, the increased space within the fluid delivery device can be used to house a larger fluid or medicament reservoir in the fluid delivery device. That is, the overall size of the fluid delivery device may remain unchanged, but a larger fluid reservoir can be included in the fluid delivery device, thereby increasing the throughput of the fluid delivery device. Compared to conventional delivery pens, the delivery pens disclosed herein can hold at least 10% more medicament than conventional delivery pens, at least 20% more medicament than conventional delivery pens, at least 25% more medicament than conventional delivery pens, at least 50% more medicament than conventional delivery pens, at least 60% more medicament than conventional delivery pens, at least 70% more medicament than conventional delivery pens, at least 80% more medicament than conventional delivery pens, at least 90% more medicament than conventional delivery pens, at least 100% more medicament than conventional delivery pens, at least 110% more medicament than conventional delivery pens, at least 120% more medicament than conventional delivery pens, at least 130% more medicament than conventional delivery pens, at least 140% more medicament than conventional delivery pens, at least 150% more medicament than conventional delivery pens, at least 160% more medicament than conventional delivery pens, at least 170% more medicament than conventional delivery pens, at least 180% more medicament than conventional delivery pens, at least 190% more medicament than conventional delivery pens, or at least 200% more medicament than conventional delivery pens.

In some embodiments, the delivery pens disclosed herein having a standard length but an increased reservoir size can hold 3.3 mL or more of medicament, 3.6 mL or more of medicament, 3.75 mL or more of medicament, 4.5 mL or more of medicament, 4.8 mL or more of medicament, 5.1 mL or more of medicament, 5.4 mL or more of medicament, 5.7 mL or more of medicament, 6.0 mL or more of medicament, 6.3 mL or more of medicament, 6.6 mL or more of medicament, 6.9 mL or more of medicament, 7.2 mL or more of medicament, 7.5 mL or more of medicament, 7.8 mL or more of medicament, 8.1 mL or more of medicament, 8.4 mL or more of medicament, 8.7 mL or more of medicament, or 9.0 mL or more of medicament.

In some embodiments, by reducing or eliminating the additional anti-rotation mechanisms through implementation of the non-circular reservoir, the footprint of the entire fluid delivery device can be reduced. That is, in some embodiments, rather than filing the increased internal space of the fluid delivery device with additional components or a larger reservoir, the exterior of the fluid delivery device can be shrunk to eliminate the increased internal space. In such embodiments, the throughput of the fluid delivery device may remain unchanged, but the overall size or length of the fluid delivery device can be reduced to improve ergonomics and transportability. Compared to conventional delivery pens, the length of the delivery pens disclosed herein can be 90% or less than the length of conventional delivery pens, 80% or less than the length of conventional delivery pens, 70% or less than the length of conventional delivery pens, 60% or less than the length of conventional delivery pens, 50% or less than the length of conventional delivery pens, 40% or less than the length of conventional delivery pens, 35% or less than the length of conventional delivery pens, 30% or less than the length of conventional delivery pens, or 25% or less than the length of conventional delivery pens.

In some embodiments, the delivery pens disclosed herein having a standard reservoir volume but reduced pen length, can be 10.0 cm or less in length, 9.5 cm or less in length, 9.0 cm or less in length, 8.5 cm or less in length, 8.0 cm or less in length, 7.5 cm or less in length, 7.0 cm or less in length, 6.5 cm or less in length, 6.0 cm or less in length, 5.5 cm or less in length, or 5.0 cm or less in length.

In some embodiments, the cross-sectional shape of the non-circular reservoir can be selected as desired to achieve a certain volume of the reservoir (if increased throughput is desired) or a certain length of the delivery pen (if a reduced pen length is desired). Generally, however, the cross-sectional shape of the non-circular reservoir will be selected to maintain the width of the delivery pen in a desirable range and to maintain the force required to advance a plunger through the reservoir in a desirable range.

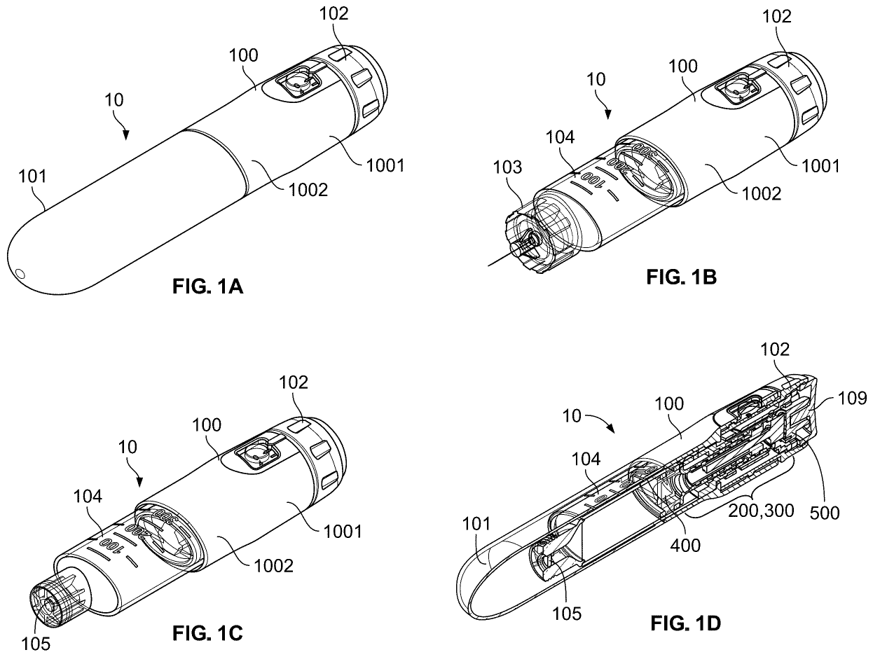

FIGS. 1A-1D illustrate perspective views of a delivery pen 10, according to aspects of this disclosure. FIG. 1A illustrates a perspective view of the delivery pen 10 with a main body 100, a cap 101, and a dose knob 102, which can be used to set and administer a dose of a desired volume, as discussed in further detail below. In some embodiments, the main body 100 comprises a circular body 1001 at a proximal side, and a non-circular body 1002 at a distal side, wherein a shape of the main body 100 transitions between the circular body 1001 and the non-circular body 1002. The non-circular body 1002 can house a non-circular reservoir 104. In some embodiments, a cross-sectional shape of the non-circular body 1002 or the non-circular reservoir 104 is elliptical, square, rectangular, triangular, or any other non-circular shape. FIG. 1B illustrates a perspective view of the delivery pen 10 with the cap 101 removed, revealing a needle 103 installed at a distal end of the reservoir 104. FIG. 1C illustrates a perspective view without the needle 103, revealing an outlet port 105 at the distal end of the reservoir 104. FIG. 1D illustrates a perspective cross-sectional view of the delivery pen 10, revealing a screw mechanism (SM) 200 or 300, a torque coupling component (TCC) 500, and a plunger 400 on the inside of the main body 100. In some embodiments, the shape of the plunger 400 matches the shape of the non-circular body 1002 to have a desired seal compression.

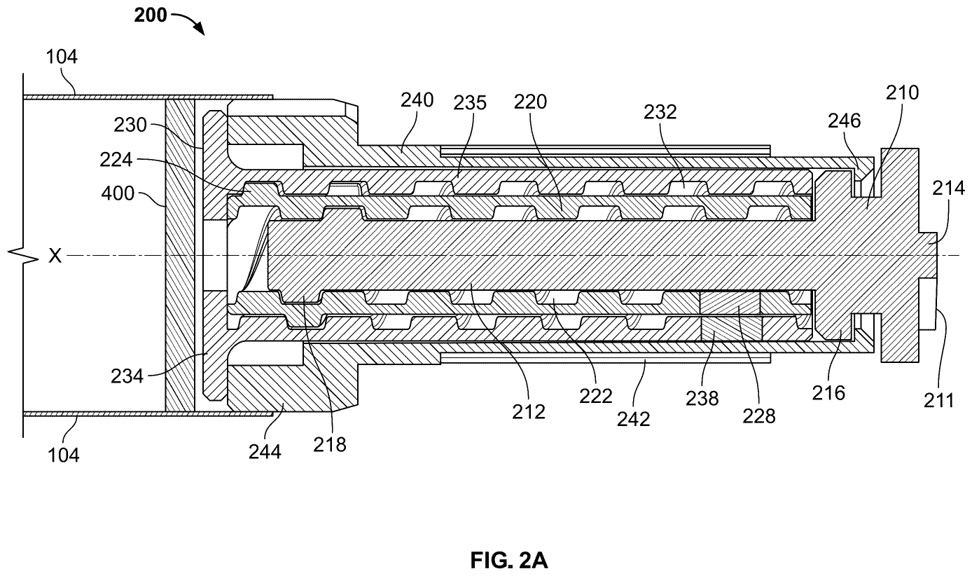

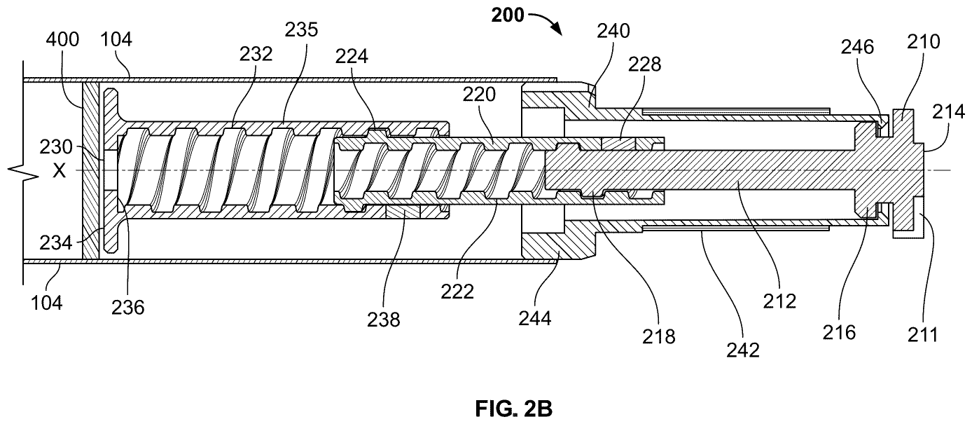

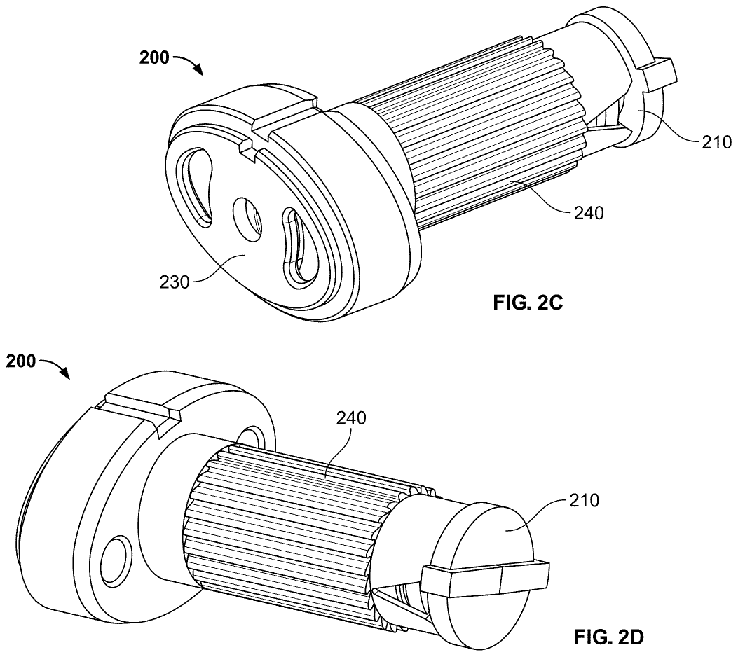

FIGS. 2A-2B illustrate cross-sectional views of the SM 200 in nested and extending positions, respectively. The SM 200 comprises three primary components: a drive shaft 210, an inner screw 220, and a pusher 230, collectively referred to as telescoping screw members. In some embodiments, each of the three primary components includes the same longitudinal centerline (e.g., the axis X). In some embodiments, the drive shaft 210 is positioned laterally within the inner screw 220, and the inner screw 220 is positioned laterally within the pusher 230. As can be seen, for example, in FIG. 2B, these components are interconnected such that a movement of the drive shaft 210 would translate the inner screw 220 and the pusher 230 in the longitudinal direction. In some embodiments, rotation of the drive shaft 210 can translate the inner screw 220 and the pusher 230. The pusher 230 can include a body 235 and a distal, non-circular end 234 extending from the body 235, where the non-circular end 234 contacts a proximal surface of a plunger 400 in the reservoir 104. In some embodiments, the non-circular end 234 and the plunger 400 are coupled together. In some embodiments, the non-circular end 234 and the plunger 400 can couple together after they are brought into contact. In some embodiments, the plunger 400 is free-floating within the reservoir 104. The non-circular end 234 of the pusher 230 can contact the plunger 400 to advance the plunger 400 through the reservoir 104 and in turn administer liquid from the reservoir 104. FIGS. 2C-2D are isometric views of the SM 200 in a nested position. In some embodiments, the SM 200 can include an outer housing 240. In the nested position, the SM 200 can be housed within the outer housing 240. Specifically, in the nested position, the inner screw 220 can be positioned within the outer housing 240, and a body 235 of the pusher 230 can be positioned within the outer housing 240.

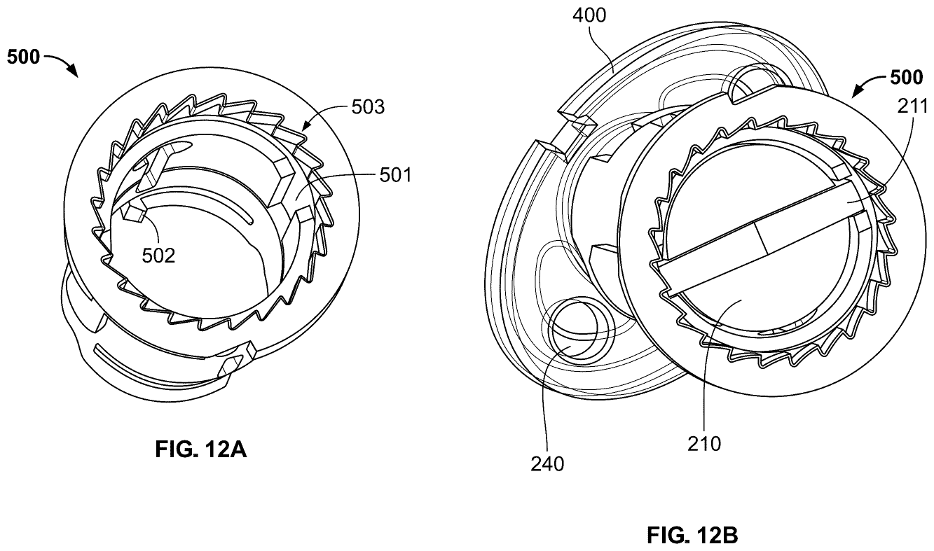

As illustrated in FIG. 2B, in some embodiments, the drive shaft 210 can include a first portion 212 that extends distally from a second portion 214. The first portion 212 comprises a mechanism for engagement with the inner screw 220 so as to allow the inner screw 220 to move longitudinally along axis X. In some embodiments, an outer surface of first portion 212 comprises a protrusion 218, wherein the protrusion 218 is in engagement with an inner surface 222 of the inner screw 220 such that rotation of the drive shaft 210, due to an applied torque, facilitates advancing of the inner screw 220 longitudinally along axis X at a first rate. In some embodiments, the protrusion 218 comprises at least a portion of a first threading. In some embodiments, the inner surface 222 of the inner screw 220 includes a corresponding threading to engage with the protrusion 218 of the drive shaft 210. The second portion 214 is provided at a proximal end of the drive shaft 210 to enable an engagement between the drive shaft 210 and the outer housing 240. In some embodiments, the second portion 214 of the drive shaft 210 contains one or more member 211 that engage with TCC 500 (also shown in FIG. 12B), such that torque applied to the dose knob 102 is transferred to the drive shaft 210. In some embodiments, the outer housing 240 comprises a proximal section 242 and a distal section 244. In some embodiments, the proximal section 242 has a circular diameter, so as to allow for nested fitting of the inner screw 220 and the pusher 230 within the proximal section 242. In some embodiments, the distal section 244 has a non-circular diameter to fit within the reservoir 104 when the reservoir 104 comprises a non-circular shape. In some embodiments, roughly half of the distal section 244 of the outer housing 240 is positioned within the reservoir 104. That is, the proximal end of the reservoir 104 is positioned between a distal end of the distal section 244 and a proximal end of the distal section 244 of the outer housing 240. As illustrated in FIG. 2B, the outer housing 240 comprises an annular circumferential protrusion 246 at a proximal end of the proximal section 242, wherein the circumferential protrusion 246 is in engagement with a member 216 of the drive shaft 210. In some embodiments, the member 216 comprises a snapping feature for a more secure engagement. The member 216 may laterally extend from the second portion 214 and curve over the circumferential protrusion 246 to create a snap fit engagement between the member 216 and the distal side of the circumferential protrusion 246. Such snap fit engagement can allow for the drive shaft 210 to rotate about, but not extend longitudinally from, the outer housing 240 due to the engagement with the circumferential protrusion 246.

In some embodiments, a distal end of the inner screw 220 comprises a mechanism for engagement with the pusher 230 to allow the pusher 230 to advance longitudinally along axis X away from a proximal end of the inner screw 220. In some embodiments, the mechanism for engagement comprises a protrusion 224 on an outer surface of the inner screw 220, wherein the protrusion 224 is in engagement with an inner surface 232 of the pusher 230 such that rotation of the inner screw 220, due to an applied torque, facilitates advancing of the pusher 230 longitudinally along axis X at a second rate. In some embodiments, the protrusion 224 comprises at least a portion of a second threading. In some embodiments, the inner surface 232 of the pusher 230 comprises an inner thread corresponding to the second threading such that movement of the inner screw 220 is applied to the pusher 230. In some embodiments, the inner surface 232 of the body 235 of the pusher 230 comprises the inner thread corresponding to the second threading such that movement of the inner screw 220 is applied to the pusher 230. In some embodiments, the corresponding threading is such that rotation of the inner screw 220 can longitudinally advance the pusher 230. As can be seen in FIGS. 2A-2B, pusher 230 comprises an inner surface 236 of a non-circular end 234, such that the inner surface 236 engages a distal end of the inner screw 220 while in the nested position, and the inner surface 236 longitudinally advances away from the distal end of the inner screw 220 when rotation is applied to the inner screw 220.

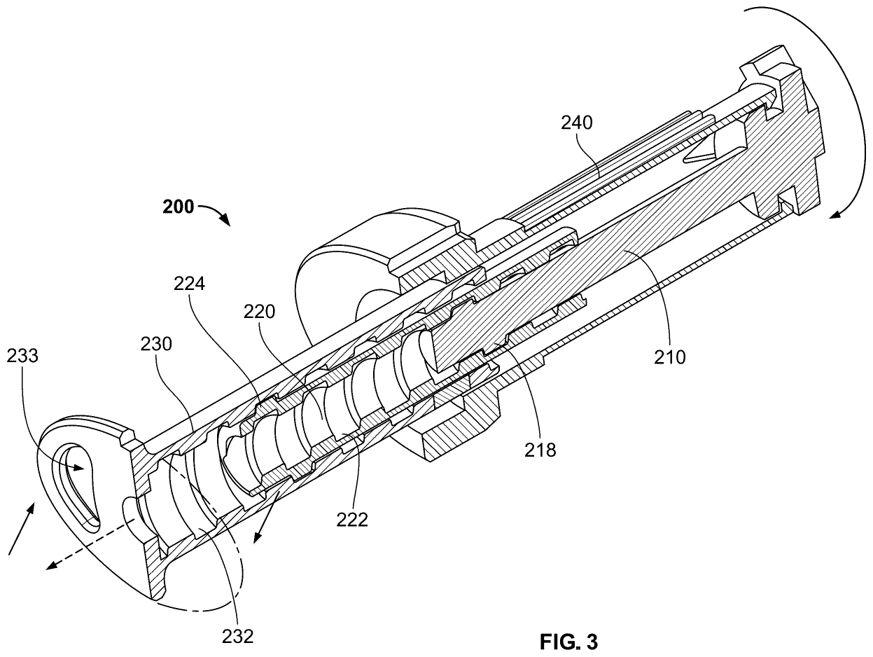

FIG. 3 shows an isometric cross-sectional view of the SM 200 in a partially extended position, illustrating a use-case scenario as torque is input at a proximal end of the drive shaft 210. In some embodiments, the first threading of the protrusion 218 on the drive shaft 210 is of a same handedness and pitch as the first threading of the protrusion 224 on the inner screw 220, and the corresponding second threading of the inner surface 222 of the inner screw 220 is of the same handedness and pitch as the corresponding second threading of the inner surface 232 of the pusher 230. In such a configuration, only one of the pusher 230 and the inner screw 220 can longitudinally advance at a given time. Such a configuration allows an opportunity for either the inner screw 220 or the pusher 230 to advance at a given time relative to the other. That is, depending on the drive shaft 210/inner screw 220 drive torque (e.g., torque required to rotate the drive shaft 210 relative the inner screw 220) relative to the inner screw 220/pusher 230 drive torque (e.g., torque required to rotate the inner screw 220 relative the pusher 230), either the drive shaft 210 will rotate with respect to the inner screw 220 or the inner screw 220 will rotate with respect to the pusher 230 at a given time, and therefore, either the inner screw 220 will longitudinally advance (due to relative rotation of the drive shaft 210 with respect to the inner screw 220) or the pusher 230 will longitudinally advance (due to relative rotation of the inner screw 220 with respect to the pusher 230) at a given time. In such a configuration, the drive shaft 220/inner screw 220 drive torque or the inner screw 220/pusher 230 drive torque can change over time. Therefore, there can be a back-and-forth shift between which of the inner screw 220 or pusher 230 is longitudinally advancing at a given time due to a change in the relative drive torques. In some embodiments, rotation of the drive shaft 210 causes the inner screw 220 to advance longitudinally (e.g., when the drive torques are such that drive shaft 210 rotates with respect to the inner screw 220). In some embodiments, rotation of the drive shaft 210 causes the inner screw 220 to rotate with the drive shaft 210, which can cause the pusher 230 to longitudinally advance with respect to the inner screw 220. The inner screw 220 and the pusher 230 can longitudinally advance in the same direction toward the reservoir. The drive torque between two members (e.g., the drive shaft 210 and inner screw 220) can be affected by the geometry of the two members or the tightness of the fit between the two members. For instance, a tighter fit between the drive shaft 210 and inner screw 220 would require a greater drive torque to rotate the drive shaft 210 relative the inner screw 220. Therefore, until this greater drive torque is achieved, the drive shaft 210 and inner screw 220 can rotate together, which can cause longitudinal advancement of the pusher 230 (when there is a lower relative drive torque of the inner screw 220/pusher 230 than the drive shaft 210/inner screw 220).

In some embodiments, the configuration where the first threading of the protrusion 218 on the drive shaft 210 is of a same handedness and pitch as the first threading of the protrusion 224 on the inner screw 220, and the corresponding second threading of the inner surface 222 of the inner screw 220 is of the same handedness and pitch as the corresponding second threading of the inner surface 232 of the pusher 230 may allow the pusher 230 to longitudinally advance at half of the displacement per revolution as the inner screw 220, or vice versa. By maintaining an equivalent thread pitch between the inner screw 220 and the pusher 230, the SM may longitudinally advance at twice the resolution, per rotation of the dose knob 102, relative to a configuration where the first threading and the second threading are of opposing handedness. The torque ratios between the inner screw 220 and the pusher 230 may be related to each component's diameter, with the smallest drive torque associated with a smaller diameter of the inner screw 220. In some embodiments, the inner screw 220 may be driven longitudinally forward first, rather than the pusher 230, based on a relative torque between the components. Generally, inner screw 220 may longitudinally advance before pusher 230 due to a smaller thread radius and therefore a lower torque requirement to be driven. In some embodiments, the pusher 230 may longitudinally advance before inner screw 220. In some embodiments, the outer housing 240 is fixed with the body of the pen 10, enabling longitudinal advancing of (a) the inner screw 220 in one direction toward a distal end of the reservoir 104 with respect to the drive shaft 210 upon rotation of the drive shaft 210, and (b) the pusher 230 toward the distal end of the reservoir 104 by relative rotation of the protrusion 224 of the inner screw 220. In this arrangement, the torque is constant and unchanging.

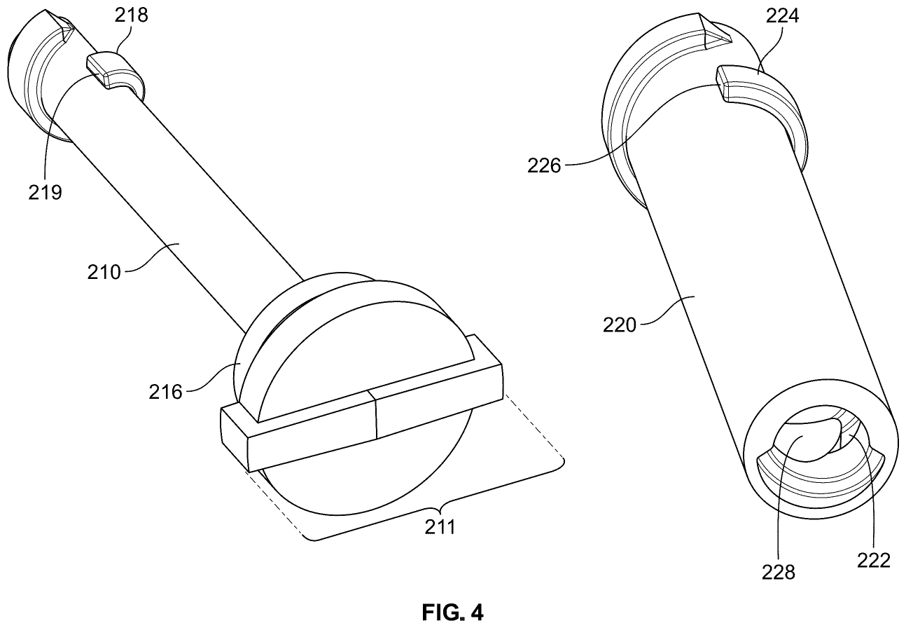

FIGS. 2A, 2B, and 4-5 illustrate stop mechanisms that may ensure that neither component of the SM 200 fully unscrews before the next component advances, described in more detail below. FIG. 4 illustrates perspective views of the drive shaft 210 and the inner screw 220, allowing for visibility of the inner surface 222 of the inner screw 220. In some embodiments, the inner screw 220 includes an interruption in the inner surface 222 to accept the stop mechanism. The stop mechanism functions regardless of which component longitudinally advances first, e.g., the inner screw 220 or the pusher 230. In some embodiments, the interruption comprises an inserted plug 228 configured to interrupt the inner surface 222, and particularly the threads of the inner surface 222. In some embodiments, the protrusion 218 of the drive shaft 210 terminates with a vertical wall 219. Rotation of the drive shaft 210 can drive the inner screw 220 longitudinally forward with respect to the drive shaft 210. As the inner screw 220 longitudinally advances along the drive shaft 210, a distance between the protrusion 218 and the plug 228 reduces until the vertical wall 219 contacts the plug 228, thereby preventing further longitudinal advancement of the inner screw 220. The plug 228 may be inserted after the inner screw 220 and the drive shaft 210 are fully screwed together during assembly.

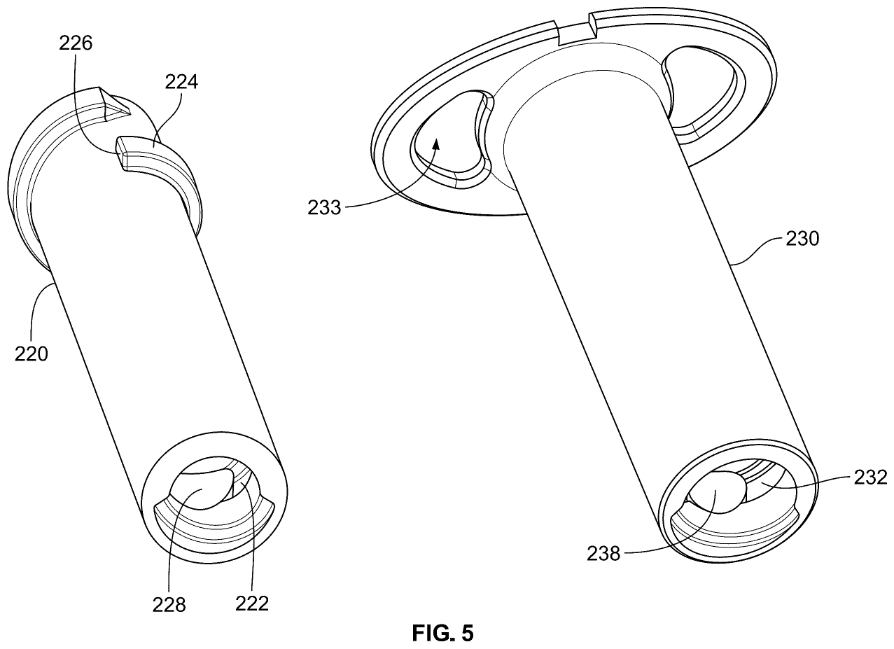

FIG. 5 illustrates perspective views of the inner screw 220 and the pusher 230, allowing for visibility of the inner surface 232 of the pusher 230. Similar to FIG. 4, the pusher 230 may include an interruption in the inner surface 232 to accept the stop mechanism. The stop mechanism, again, functions regardless of which component longitudinally advances first, e.g., the inner screw 220 or the pusher 230. In some embodiments, the interruption comprises an inserted plug 238 configured to interrupt the inner surface 232, and particularly the threads of the inner surface 232. In some embodiments, the protrusion 224 of the inner screw 220 terminates with a vertical surface 226. Rotation of the drive shaft 210 can rotate the inner screw 220. Rotation of the inner screw 220 can drive the pusher 230 longitudinally forward with respect to the inner screw 220. As the pusher 230 longitudinally advances along the inner screw 220, a distance between the protrusion 224 and the plug 238 reduces until the vertical surface 226 contacts the plug 238, thereby preventing further longitudinal advancement of the pusher 230. The plug 238 may be inserted after the pusher 230 and the inner screw 220 are fully screwed together during assembly.

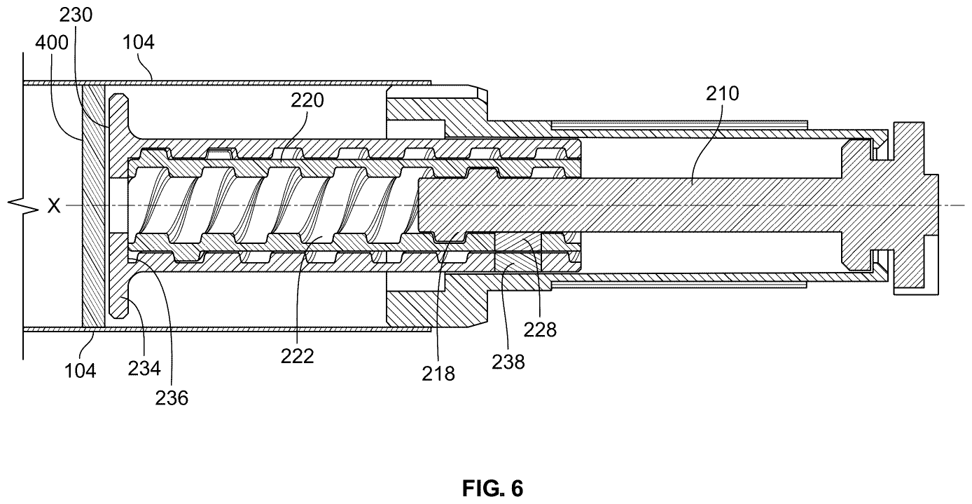

In general, the expected behavior based on nominal geometry is for components with smaller diameter to extend first due to lower torque requirements. FIG. 6 illustrates a scenario of the inner screw 220 advancing first, rather than the pusher 230. For instance, due to the relative drive torques, the drive shaft 210 can rotate with respect to the inner screw 220 while the inner screw 220 does not rotate with respect to the pusher 230, causing the inner screw 220 to longitudinally advance (and carry the pusher 230 with it). Here, the distal end of the inner screw 220 will remain in contact with the inner surface 236 of the end 234 of the pusher 230 as the two components translate or longitudinally advance with respect to the drive shaft 210, as shown in FIG. 6. Separation of the inner screw 220 and the pusher 230 occurs once the vertical wall 219 of the protrusion 218 of the drive shaft 210 collides with the side wall of the plug 228 in the inner surface 222 of the inner screw 220, initiating longitudinal advancement of the pusher 230 along the second threading of the pusher 230. In some embodiments, as the drive shaft 210 rotates, the inner screw 220 longitudinally advances with respect to the drive shaft 210 until the vertical wall 219 of the protrusion 218 of the drive shaft 210 collides with the side wall of the plug 228 in the inner surface 222 of the inner screw 220. In some embodiments, after the vertical wall 219 of the protrusion 218 of the drive shaft 210 collides with the side wall of the plug 228 in the inner surface 222 of the inner screw 220, rotation of the drive shaft 210 can cause rotation of the inner screw 220, which in turn initiates longitudinal advancement of the pusher 230 with respect to the inner screw 220. Specifically, in some embodiments, contact between the plug 228 in the inner surface 222 of the inner screw 220 and the vertical wall 219 of the protrusion 218 of the drive shaft 210 increases the drive torque of the inner screw 220/drive shaft 210. As an example, contact between the protrusion 218 and the plug 228 can tighten the fit between the inner screw 220 and the drive shaft 210. Such tightening can cause the drive torque of the inner screw 220/drive shaft 210 to be greater than the drive torque of the pusher 230/inner screw 220. Therefore, with continued rotation of the drive shaft 210, the drive shaft 210 and inner screw 220 can rotate together, and the relative rotation of the inner screw 220 with respect to the pusher 230 can longitudinally advance the pusher 230 relative the inner screw 220. Longitudinal advancement of the pusher 230 along the second threading will continue until the plunger 400 displaces all medicament in the reservoir 104. In some embodiments, longitudinal advancement of the pusher 230 along the second threading will continue until a distal end of the plunger 400 reaches a distal end of the reservoir. In some embodiments, longitudinal advancement of the pusher 230 along the second threading will continue until the vertical surface 226 of the protrusion 224 of the inner screw 220 collides with the side wall of the plug 238 in the inner surface 232 of the pusher 230. FIG. 2B shows an example of the pusher 230 longitudinally advanced with respect to the inner screw 220, which is longitudinally advanced with respect to the drive shaft 210.

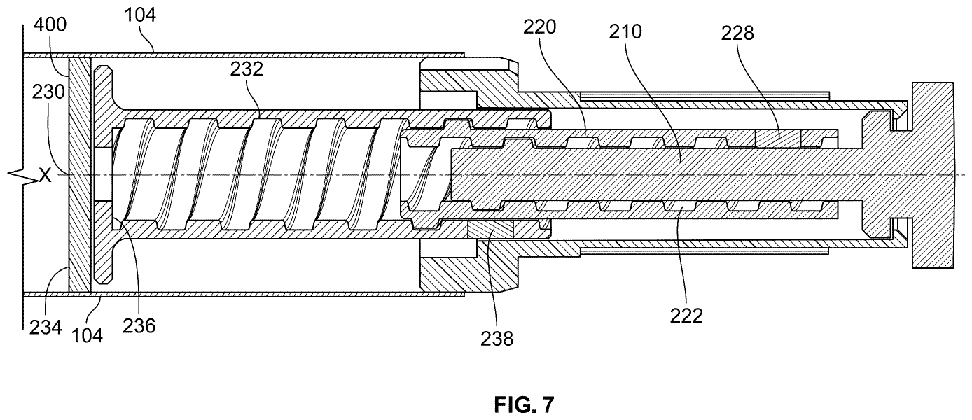

In some embodiments, as shown in FIG. 7, the pusher 230 may be longitudinally advanced first before the inner screw 220. For instance, due to the relative drive torques, the inner screw 220 can rotate with respect to the pusher 230 while the drive shaft 210 does not rotate with respect to the inner screw 220, causing the pusher 230 to longitudinally advance. In some embodiments, the pusher 230 comprises an interruption in the inner surface 232. As illustrated in FIGS. 6-7, the pusher 230 comprises the inserted plug 238 configured to interrupt the inner surface 232, and particularly the threads of the inner surface 232. The plug 228 may be inserted after the pusher 230 and the inner screw 220 are fully screwed together during assembly. As discussed above, the protrusion 224 of the inner screw 220 terminates with the vertical surface 226. In this configuration, the pusher 230 may longitudinally advance with respect to the inner screw 220 until the side wall of the plug 238 collides with the vertical surface 226 of the protrusion 224 of the inner screw 220, initiating advancement along a second threading of the inner screw 220. In some embodiments, after the vertical surface 226 of the protrusion 224 of the inner screw 220 collides with the side wall of the plug 238 in the inner surface 232 of the pusher 230, the drive shaft 210 can rotate with respect to the inner screw 220, which in turn initiates longitudinal advancement of the inner screw 220 with respect to the drive shaft 210. Specifically, in some embodiments, contact between the plug 238 in the inner surface 232 of the pusher 230 and the vertical wall 226 of the protrusion 224 of the inner screw 220 increases the drive torque of the pusher 230/inner screw 220. As an example, contact between the protrusion 224 and the plug 238 can tighten the fit between the inner screw 220 and the pusher 230. Such tightening can cause the drive torque of the pusher 230/inner screw 220 to be greater than the drive torque of the inner screw 220/drive shaft 210. Therefore, with continued rotation of the drive shaft 210, the drive shaft 210 can rotate with respect to the inner screw 220, and the relative rotation of the drive shaft 210 with respect to the inner screw 220 can longitudinally advance the inner screw 220 relative the drive shaft 210. Longitudinal advancement of the inner screw 220 along the second threading will continue until the plunger 400 displaces all medicament in the reservoir 104. In some embodiments, longitudinal advancement of the inner screw 220 along the second threading will continue until a distal end of the plunger 400 reaches a distal end of the reservoir 104. In some embodiments, longitudinal advancement of the inner screw 220 along the second threading will continue until the vertical wall 219 of the protrusion 218 of the drive shaft 210 collides with the side wall of the plug 228 in the inner surface 222 of the inner screw 220. FIG. 2B shows an example of the pusher 230 longitudinally advanced with respect to the inner screw 220, which is longitudinally advanced with respect to the drive shaft 210.

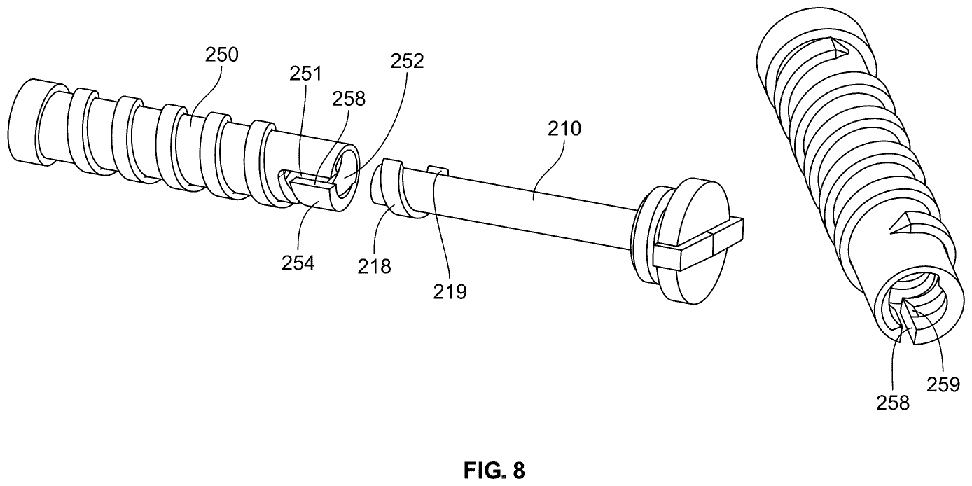

FIG. 8 shows a second embodiment of a stop mechanism between the drive shaft 210 and an inner screw 250, wherein the inner screw 250 is an alternative embodiment to the inner screw 220. A proximal end of the inner screw 250 may comprise a cantilever beam 254, and a leading surface of the cantilever beam 254 comprises a vertical surface 258. An inner surface 252 of the inner screw 250 may comprise one continuous thread that is only interrupted by a slot 251 and a ramp 259 as replacements to the plug 228 for interrupting the inner surface 252. During assembly, the drive shaft 210 is threaded into the inner screw 250, and the protrusion 218 is configured to engage and deform the cantilever beam 254 until the vertical wall 219 of the protrusion 218 of the drive shaft 210 clears the vertical surface 258 of the inner screw 250. The drive shaft 210 and the inner screw 250 can now be assembled in the collapsed position, similar to the configuration shown in FIG. 2A. Once the device is assembled and torque is applied to the drive shaft 210, the inner screw 220 may longitudinally advance along the drive shaft 210 until the vertical wall 219 of the drive shaft 210 collides with the vertical surface 258 of the cantilever beam 254. As discussed in the previous embodiments, after contact between the vertical wall 219 of the drive shaft 210 and the stop mechanism of the inner screw 250, in this case the vertical surface 258 of the cantilever beam 254, rotation of the drive shaft 210 can cause rotation of the inner screw 250. The pusher 230 is then allowed to start longitudinally advancing until the reservoir 104 is empty of medicament. Alternative designs for a stop mechanism for the drive shaft 210 and the inner screw 250 could be a swage, heat stake or clip after screwing the parts together.

In accordance with an example embodiment, a length of the SM 200 is dimensioned such that, when the telescoping screw members are all nested or collapsed, the inner screw 220 and the body 235 of the pusher 230 are all contained in the housing 240. In some embodiments, each of the telescoping screw members is a desired length. The desired length corresponds to a potential extension length of the pusher the 230 relative to the outer housing the 240.

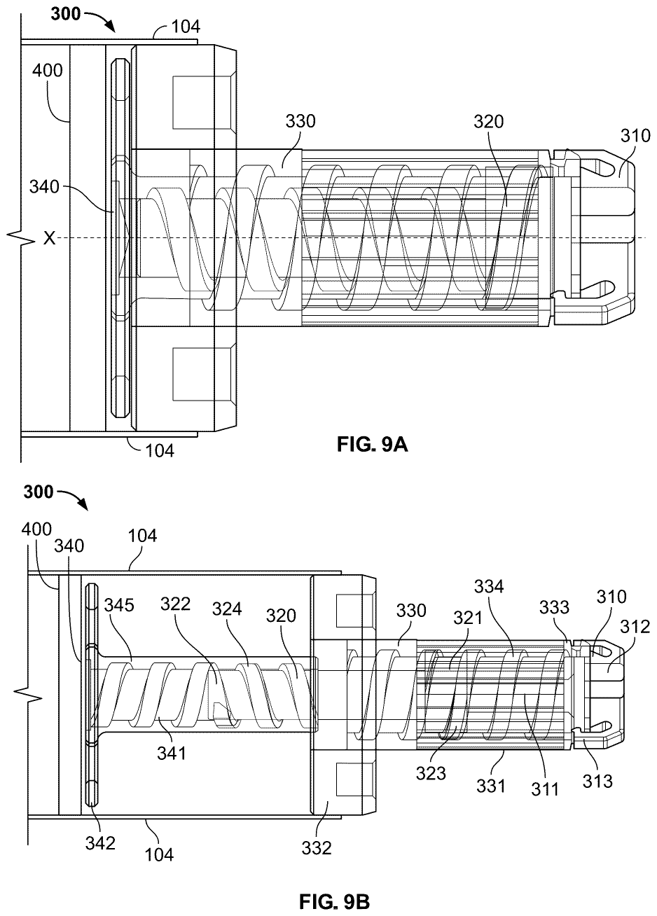

FIGS. 9A-9B illustrate transparent views of an SM 300 in nested and extended positions, respectively, according to some aspects of this disclosure. In the nested position, as illustrated in FIG. 9A, the SM 300 comprises four primary components aligned about a longitudinal axis X: a drive shaft 310, an inner screw 320, an outer screw 330, and a pusher 340, collectively referred to as telescoping screw members. The outer screw 330 can be a housing to at least partially contain the drive shaft 310, inner screw 320, and pusher 340. In some embodiments, each of the four primary components includes the same longitudinal centerline (e.g., the axis X). In some embodiments, the drive shaft 310 is positioned laterally within the inner screw 320, the inner screw 320 is positioned at least partially laterally within the pusher 340, and the pusher 340 is positioned laterally within the outer screw 330. The extended position illustrated in FIG. 9B provides a clearer indication of how each component is connected. Specifically, drive shaft 310 is shown to have a first portion 311 that extends longitudinally from a second portion 312. While in the nested position, the first portion 311 is internal to the inner screw 320, wherein an inner surface of inner screw 320 is engaged with the first portion 311 such that rotation of the drive shaft 310 induces rotation of the inner screw 320. In some embodiments, the inner surface of inner screw 320 comprises an inner keying feature that corresponds with an outer keying feature on an outer surface of the first portion 311 to facilitate this engagement. The engagement between the drive shaft 310 and the inner screw 320 can be such that rotation of the drive shaft causes rotation of the inner screw 320 (e.g., the inner screw 320 does not rotate with respect to the drive shaft 310), and the inner screw 320 is longitudinally advanceable with respect to the drive shaft 310. For instance, in some embodiments, outer keying feature of the first portion 311 of the drive shaft 310 can longitudinally slide within the inner keying feature of the inner screw 320 but cannot rotate out of engagement with the inner keying feature of the inner screw 320.

The second portion 312 is provided at a proximal end of the drive shaft 310, wherein the second portion 312 is externally engaged with the outer screw 330. In some embodiments, outer screw 330 comprises a proximal section 331 and a distal section 332. In some embodiments, proximal section 331 has a circular diameter. In some embodiments, distal section 332 has a non-circular diameter. In some embodiments, roughly half of the distal section 332 of the outer screw 330 is positioned within the reservoir 104. That is, the proximal end of the reservoir 104 is positioned between a distal end of the distal section 332 and a proximal end of the distal section 332 of the outer screw 330. As illustrated in FIG. 9B, outer screw 330 comprises an annular indent 333 at a proximal end of proximal section 331, wherein the indent 333 is configured to engage one or more member 313 extending from second portion 312 of the drive shaft 310. In some embodiments, one or more member 313 comprises a snapping feature. The one or more member 313 laterally extend from second portion 312 and curve over a distal end of outer screw 330 to be seated in the indent 333 to create a snap fit engagement between the member 313 and a distal side of a circumferential protrusion formed by the indent 333. Such snap fit engagement can allow the drive shaft 310 is to rotate about, but not extend longitudinally from, outer screw 330 due to the engagement with indent 333.

The outer screw 330 further comprises an inner surface 334 configured to engage the inner screw 320. As illustrated in FIG. 9B, inner screw 320 comprises a wide section 321 and a narrow section 322, wherein an outer diameter of wide section 321 is greater than an outer diameter of narrow section 322. In some embodiments, wide section 321 comprises a wide outer surface 323, wherein wide outer surface 323 is configured to engage inner surface 334 of outer screw 330 such that rotation of inner screw 320, due to applied torque, facilitates advancing of inner screw 320 longitudinally along axis X at a first rate. In some embodiments, wide outer surface 323 of the inner screw 320 comprises a first threading and inner surface 334 of the outer screw 330 comprises a corresponding second threading. In some embodiments, the corresponding threading allows for the longitudinal advancement of the inner screw 320 along the outer screw by rotation of the inner screw 320.

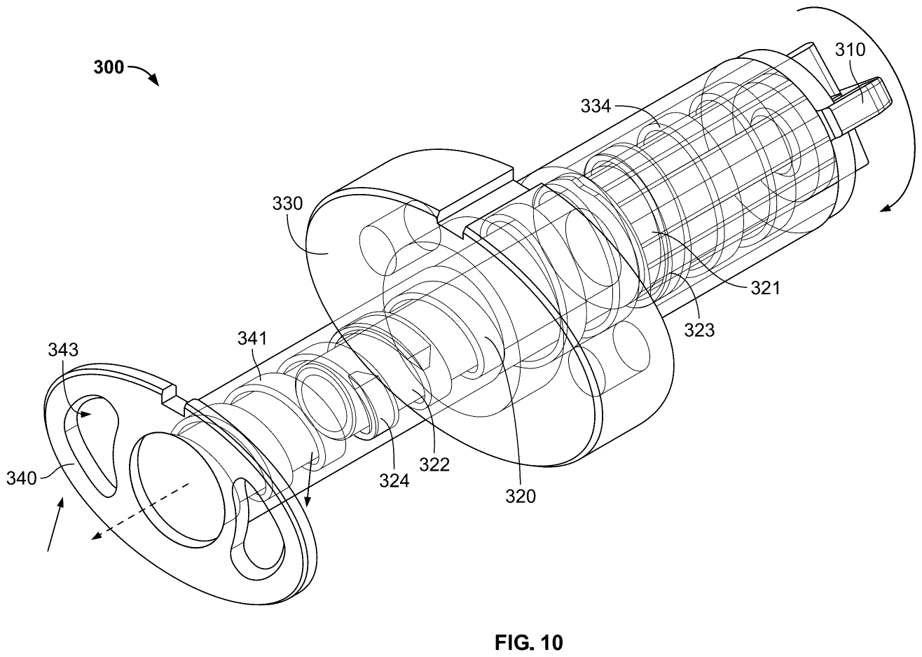

With reference to FIG. 10, in some embodiments, narrow section 322 of the inner screw 320 comprises a narrow outer surface 324, wherein narrow outer surface 324 is configured to engage an inner pusher surface 341 of pusher 340. In some embodiments, narrow outer surface 324 of the inner screw 320 is configured to engage inner pusher surface 341 of the pusher 340 such that rotation of inner screw 320, due to applied torque, facilitates advancing of the pusher 340 longitudinally along axis X. In some embodiments, narrow outer surface 324 of the inner screw 320 comprises a first threading and the inner pusher surface 341 of pusher 340 comprises a corresponding second threading. In some embodiments, the corresponding threading allows for the longitudinal advancement of the pusher 340 along the inner screw 320 by rotation of the inner screw 320. In some embodiments, the first threading of the narrow outer surface 324 of the inner screw 320 is of an opposing handedness to and same pitch as the first threading of wide outer surface 323 of the inner screw 320, and the corresponding second threading of inner pusher surface 341 of pusher 340 is of an opposing handedness to and same pitch as the corresponding second threading of inner surface 334 of the outer screw 330. In such a configuration, the pusher 340 will longitudinally advance with respect to the inner screw 320 simultaneously with the inner screw 320 longitudinally advancing with respect to the drive shaft 310 and outer screw 330. In such a configuration, the inner screw 320 can rotate with respect to the outer screw 330 and the pusher 340 simultaneously with advancing longitudinally with respect to the drive shaft 310 and the outer screw 330. This configuration causes the inner screw 320 to longitudinally advance the same amount at the same time along the outer screw 330 as the pusher 340 longitudinally advancing along the inner screw 320. The inner screw 320 and pusher 340 advance in the same direction toward the reservoir. In some embodiments, outer screw 330 is fixed with the body of the pen. The embodiment of FIGS. 9A-10 can enable simultaneous advancing of (a) the inner screw 320 toward the reservoir with respect to the outer screw 330 upon rotation of the drive shaft 310 (by relative rotation of the wide section 321 of the inner screw 320 with respect to the outer screw 330), and (b) the pusher 340 toward the reservoir by relative rotation of the narrow section 322 of inner screw 320 with respect to the pusher 340. In this arrangement, the torque is constant and unchanging.

In accordance with an example embodiment, a length of SM 300 is dimensioned such that, when the telescoping screw members (e.g., drive shaft 310, inner screw 320, and pusher 340) are all nested or collapsed, the inner screw 320 and a body 345 of the pusher 340 are contained in the outer screw 330. In some embodiments, each of the telescoping screw members is a desired length. The desired length corresponds to a potential longitudinal extension length of the pusher 340 relative to the outer screw 330. Because the pusher 340 and inner screw 320 longitudinally advance simultaneously, a non-circular end 342 of the pusher 340 can reach the distal end of the reservoir 104 without the proximal end of the pusher 340 reaching the distal end of the inner screw 320, such that the pusher 340 and inner screw 320 cannot unthread.

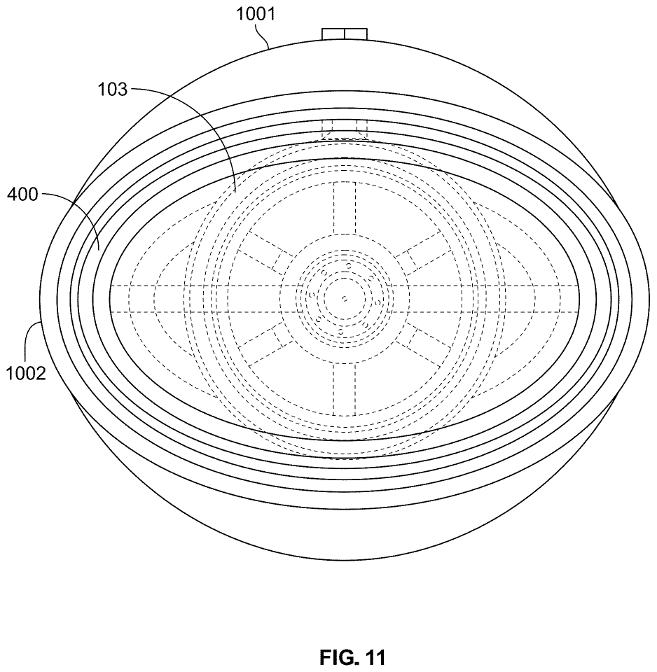

In some embodiments, pusher 340 comprises the non-circular end 342 at a distal end of the body 345 in engagement with plunger 400 as illustrated in FIG. 1D. In some embodiments, non-circular end 342 comprises a shape that corresponds to an inner shape of the reservoir 104. The shape of non-circular end 342, which corresponds to the shape of non-circular body 1002 as illustrated in FIG. 11, constrains the pusher 340 within reservoir 104 to prevent its rotation such that torque applied to inner screw 320 cannot be transferred to pusher 340, rather the pusher 340 is facilitated to extend longitudinally along axis X from narrow section 322 of inner screw 320 at a second rate. In some embodiments, the first rate of advancement of the inner screw 320 and the second rate of longitudinal advancement of the pusher 340 can be modified by changing the pitch of corresponding threads between at least one of (a) wide section 321 of inner screw 320 and outer screw 330 or (b) narrow section 322 of inner screw 320 and pusher 340.

FIG. 11 illustrates a front view of a delivery pen, according to some aspects of this disclosure. In some embodiments, the pusher 230 comprises a non-circular end 234 (shown in FIG. 7), which corresponds to the shape of the non-circular body 1002 and may be in engagement with the plunger 400. In some embodiments, the non-circular end 234 comprises a shape that corresponds to an inner shape of the reservoir 104. The shape of the non-circular end 234 constrains the pusher 230 within the reservoir 104 to prevent its rotation such that torque applied to the inner screw 220 cannot be transferred to the pusher 230, rather the pusher 230 is facilitated to extend longitudinally along axis X at a second rate. In some embodiments, the first rate of advancement of the inner screw 220 and the second rate of advancement of the pusher 230 can be modified by changing the pitch of corresponding threads between at least one of (a) the drive shaft 210 and the inner screw 220, or (b) the inner screw 220 and the pusher 230.

FIGS. 12A-12B illustrate perspective views of the TCC 500 alone and assembled with the SM 200, respectively, according to some aspects of this disclosure. While engagement between the TCC 500 and the SM 200 is particularly discussed below, it should be appreciated that the same description similarly applies to engagement between the TCC 500 and the SM 300. In some embodiments, the TCC 500 is driven by other rotational components, such as the dose knob 102 of the delivery pen 10 when a button (not shown) or other delivery method at a proximal end of the delivery pen 10 is pressed. In some embodiments, the TCC 500 comprises one or more of an axial slot 501 configured to mate with one or more of the member 211 of the drive shaft 210 (or in the case of SM 300, member 313 of the drive shaft 310) such that torque applied to the TCC 500 is transferred to the SM 200. Rotation of the drive shaft 210 facilitates axial, slidable advancement through the one or more of the axial slot 501 until the member 211 of the drive shaft 210 contacts the bottom of the slots 501 of the TCC 500, preventing disassembly of the drive shaft 210 and the TCC 500. In some embodiments, one or more of a ratchet arm 502 is positioned on an internal surface of the TCC 500. During dose setting, the TCC 500 is pulled back or pushed forward along the drive shaft 210, by rotation of the dose knob 102, to set a desired dose size. In some embodiments, the TCC 500 comprises a plurality of teeth 503 at a proximal end. The teeth 503 interact with corresponding ratchet arms of a double-clicker component (not shown) sandwiched between the TCC 500 and the dose knob 102. In some embodiments, the teeth 503 produce an audible and tactile “click” noise when the user dials, corrects, or administers a dose. The double-clicker component facilitates load transfer during pressing of the button at the proximal end of the dose knob 102 to move the TCC 500.

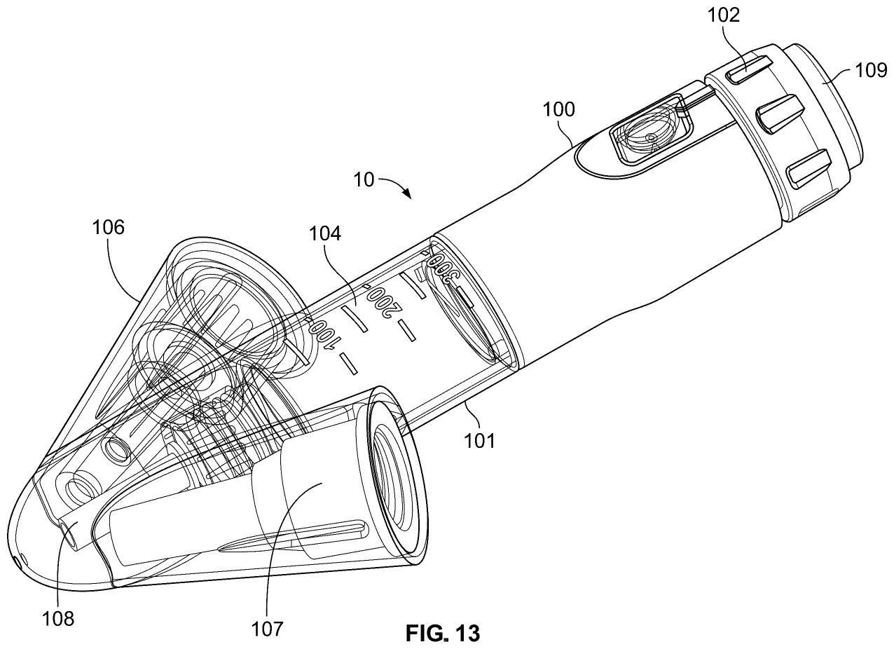

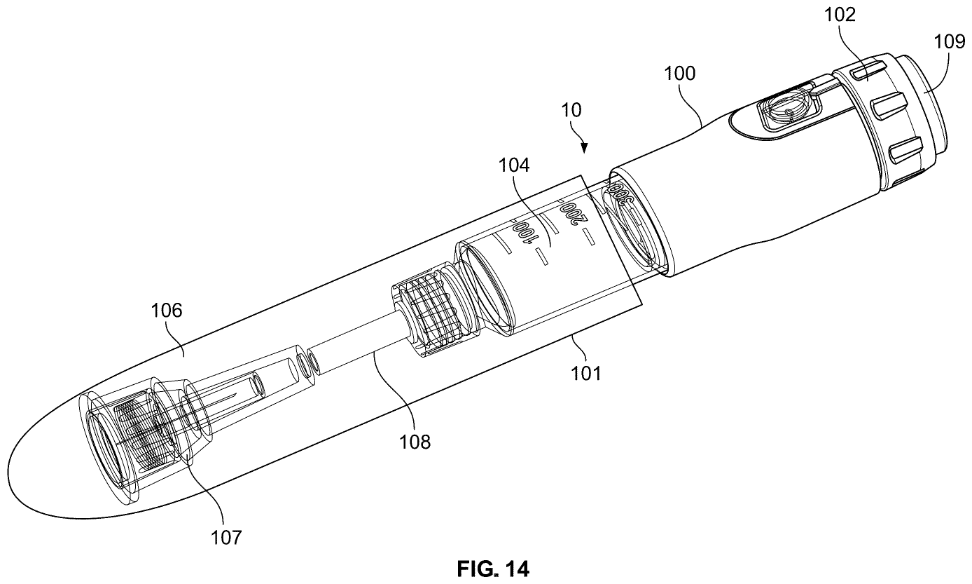

Due to the reduced size and form factor of the SM 200, 300 and reservoir 104, the delivery pen 10 can be designed for additional features that incorporate further functionality. In some embodiments, with reference to FIG. 13, the delivery pen 10 further comprises one or more storage compartment 106. In some embodiments, the one or more storage compartment 106 may have an arrow shape. The storage compartment 106 can be designed such that the delivery pen 10 retains user-friendly dimensions. In some embodiments, the storage compartment 106 is configured to fit one or more additional pen needle assembly 107, wherein each of the one or more pen needle assembly further comprises a needle shield 108. In some embodiments, the storage compartment 106 further comprises one or more of a press-in cap or hinge (not shown) to cover the one or more additional pen needle assembly 107. In some embodiments, the storage compartment 106 is configured to fit additional medication for delivery (not shown). In some embodiments, the storage compartment 106 is formed as part of the cap 101 so as to extend a longitudinal length of cap 101 at a distal end of the delivery pen 10, as shown in FIG. 14. In some embodiments, the storage compartment 106 extends longitudinally at a proximal end of the delivery pen (not shown).

In some embodiments, the additional features that may be incorporated as a result of the reduced size are electronic components or modules. In some embodiments, one or more electronic components or modules may be coupled to the delivery pen 10. In some embodiments, the one or more electronic components may be attached to the delivery pen 10 within an electronic storage compartment (not shown). In some embodiments, the one or more electronic components may be electrically and communicatively coupled with each other within the electronic storage compartment. In some embodiments, the electronic components may comprise a dose capture device, including a microcontroller electrically coupled with other electronic components. In some embodiments, the one or more electronic components may include a battery, a gyroscope, a force sensor, a controller, a network interface hardware, and/or other electronic modules. In some embodiments, the battery may be a coin cell battery. In some embodiments, the battery is specified such that it can provide power to run the systems of the dose capture device for a determined life of the dose capture device. In some embodiments, the battery is a rechargeable battery that is connected to a charging port (not shown).

In some embodiments, the delivery pen 10 may be disposable. In some embodiments, the reservoir 104 may contain medication supply to be administered to a patient. In some embodiments, the reservoir 104 may have a volume capacity of up to 6 mL. In some embodiments, as illustrated in FIG. 3, the pusher 230 optionally comprises one or more of an aperture 233. In some embodiments, the one or more of the aperture 233 allows for air to flow around a perimeter to fill in space left void after the plunger 400 is displaced. In some embodiments, the pusher 230 comprises one or more of the aperture 233 in the non-circular end 234. The aperture features can facilitate a higher rate of delivery by providing a more open flow path. The one or more of the aperture 233 functions as an assembly aid to enable pressing on the outer housing 240 to press into the body 100 of the delivery pen 10 which may help reduce pressing and possible bending forces on the pusher 230 that would cause damage or failure. In some embodiments, as illustrated in FIG. 10, the pusher 340 optionally comprises one or more aperture 343, analogous to the aperture 233 of the pusher 230.

In some embodiments, the main body 100 may provide a gripping surface for a user to grip when administering medication to a patient. In some embodiments, the delivery pen includes the dose knob 102 positioned at a proximal end of the main body 100. The dose knob 102 may be rotatable with respect to the main body 100 of the delivery pen 10. In some embodiments, the user may rotate the dose knob 102 to selectively set a desired volume of a dose of medication to be injected to a patient. In some embodiments, rotation of the dose knob 102 in a first direction may adjust and reduce the amount of rotation achievable by the drive shaft 210, thereby decreasing the volume of a dose of medication to be administered with the delivery pen 10. In some embodiments, rotation of the dose knob 102 in a second direction may adjust and increase the amount of rotation achievable by the drive shaft 210, thereby increasing the volume of a dose of medication to be administered with the delivery pen 10. In some embodiments, a button 109 may be coupled to a proximal end of the dose knob 102. A user may apply an axial force to the button 109 to depress the button 109 and the dose knob 102 axially toward the proximal end of the main body 100, thereby activating the SM 200, 300 to administer a dose of medication from the delivery pen 10. In some embodiments, a delivery method using at least one of a motor and a mechanical automation system may be coupled to a proximal end of the dose knob 102, such that the delivery method depresses the dose knob 102 axially toward the main body 100 to activate the SM 200 and administer a dose of medication from the delivery pen 10.

It is to be understood that the dose knob 102 can control the telescoping screw members to move the telescoping screw members incrementally from the fully retracted to the fully extended positions shown to move the plunger 400 and to deliver respective designated dose amounts of fluid from the reservoir 104. The TCC 500 rotates the drive shaft 210, 310 on the SM 200, 300. The TCC 500 can have different configurations. For example, the TCC 500 can also be in the form of a ratchet indexing mechanism or other indexing mechanism that precisely rotates the drive shaft 210, 310 by a mechanically controlled amount. The TCC 500 and the drive shaft 210 of the SM 200 (or drive shaft 310 of the SM 300) can be mounted with respect to each other.

The configuration of the SM 200, 300 components with respect to the reservoir 104 and the plunger 400 realizes a number of advantages. For example, having the SM 200, 300 mounted at a proximal end of the reservoir 104 and having a nested configuration that does not extend into the reservoir 104 until the drive shaft 210, 310 is rotated optimizes use of the reservoir 104 for fluid delivery instead of having to accommodate pre-delivery pen components. In addition, the overall length of the reservoir 104 can be substantially the same as the length of the cap 101 and main body 100 combined, with the addition of a small amount of headspace to accommodate the TCC 500 connection to the drive shaft 210, 310. Thus, the overall footprint of the SM 200, 300 is minimized as well as the longitudinal axis dimension of the overall delivery pen 10. The use of the plunger 400 and the SM 200, 300 design also minimizes contact of the SM 200, 300 with the fluid being delivered to ensure biocompatibility between the fluid and the main body 100. The example embodiments described herein employ nested telescoping screws of appropriate size and thread configuration to achieve a controlled movement of the non-circular reservoir plunger 400. Screw-thread technology is well-defined and understood, and is capable of repeatable, powerful motion. When driven with an appropriate resolution-controlled motion by the TCC 500, the telescoping screw members can provide accurate movement under virtually all environmental conditions. Further, the drive mechanism (e.g., the SM 200, 300) the does not affect the basic volume of the reservoir 104 where the drug resides, thus having no impact on any compatibility issues.

The reservoir 104 can be configured to be durable, that is, not removable but rather preinstalled within the main body 100. The reservoir 104 can be similar in materials to a syringe and associated stopper. The reservoir 104 can be prefilled and the SM 200, 300 initially in a retracted position. The reservoir 104 can be configured for filling by a user with a syringe, or by using a filling station that fluidically couples to the outlet port 105.

The driving and delivery mechanisms of the delivery pen 10 should be understood by a person having ordinary skill in the art and are, therefore, not discussed in detail herein. Generally, however, depression of the button and the dose knob 102 in the distal direction injects the dosed medication via the SM 200, 300 and the plunger 400 through the reservoir 104, which is contained within the delivery pen 10. The distal movement of the plunger 400 within the reservoir 104 causes medication to be forced into the needle 103. The reservoir 104 may be sealed by a septum (not shown), which may be punctured by the needle. In some embodiments, the pen needle may be screwed onto the reservoir 104, although other attachment means may be used. It should be appreciated that the foregoing description is merely one representative example of the delivery pen 10, and other designs for the delivery pen 10 are contemplated herein. In some embodiments, the dose knob 102 is coupled to an electronic or “smart” dose capture device. In some embodiments, “smart” dose capture device includes wireless functionality to enable transfer of dose information to an external wireless device.

The technical solution of the example embodiments is based on a basic screw-drive mechanism where lifting torque is a function of applied axial load (force or pressure), thread pitch, friction parameters, and diameter. In some cases, the equations may be further expanded to capture the full details of thread geometry such as flank and lead angle, and many other special parameters. Industry standard sizes for ACME threads can generally be used to adjust the balance of lifting torque, power required, efficiency, and other functional parameters such as smoothness of operation and cost. Other thread forms can also be used, such as Buttress threads, to accurately control load-transfer, and minimize dosing errors. Each screw design may affect torque; therefore, changes should be made in a manner that is congruent with the capabilities of the motor and gearbox or index drive sub-system.

There are no delivery pens that use this type of mechanism. This design brings significant space while trading some mechanical losses. The space savings open up significant design space for drug delivery pens with high delivery accuracy potential. The design of the example embodiments of the present disclosure can be complemented with a ratcheting or indexing drive transmission to further improve the motion resolution, resulting in accurate drug delivery.

Non-limiting embodiments of the present disclosure are set out in the following clauses:
Clause 1. A delivery pen comprising: a reservoir comprising a non-circular shape; a plunger positioned in the reservoir; and a screw mechanism at least partially inserted within the reservoir, the screw mechanism comprising: a drive shaft comprising one or more protruding member at a proximal end and an elongated member extending longitudinally from the proximal end; an inner screw concentrically engaging the elongated member; an outer housing comprising a circular section rotatably engaging the one or more protruding member and a non-circular section sized to fit within the reservoir; and a pusher disposed between the inner screw and the outer housing to linearly translate the plunger so as to dispense a medication from the reservoir.
Clause 2. The delivery pen of clause 1, further comprising: a first protrusion toward a distal end of an outer surface of the elongated member, and a first interruption toward a proximal end of an inner surface of the inner screw, wherein the first protrusion is configured to engage the first interruption as the inner screw longitudinally advances along the drive shaft.
Clause 3. The delivery pen of clause 1 or clause 2, wherein the first protrusion and the first interruption are positioned such that they are most separated when the drive shaft is fully nested within the inner screw.
Clause 4. The delivery pen of any one of clauses 1-3, wherein the first protrusion comprises a thread and the first interruption comprises an inserted plug.
Clause 5. The delivery pen of any one of clauses 1-4, further comprising: a second protrusion toward a distal end of an outer surface of the inner screw, and a second interruption toward a proximal end of an inner surface of the pusher, wherein the second protrusion is configured to engage the second interruption as the pusher longitudinally advances along the inner screw.
Clause 6. The delivery pen of any one of clauses 1-5, wherein the second protrusion and the second interruption are positioned such that they are most separated when the inner screw is fully nested within the pusher.
Clause 7. The delivery pen of any one of clauses 1-6, wherein the second protrusion comprises a thread and the second interruption comprises an inserted plug.
Clause 8. The delivery pen of any one of clauses 1-7, wherein: the drive shaft comprises a first threading at a distal end of an outer surface of the elongated member; the inner screw comprises a second threading on an inner surface of the inner screw, wherein the first threading and the second threading are engaged such that the inner screw can be longitudinally advanced by rotation of the drive shaft; the inner screw comprises a third threading at a distal end of an outer surface of the inner screw; and the pusher comprises a fourth threading on an inner surface of the pusher, wherein the third threading and the fourth threading are engaged such that the pusher can be longitudinally advanced by rotation of the inner screw.
Clause 9. The delivery pen of any one of clauses 1-8, wherein: the first threading and the third threading are of a same handedness; and the second threading and the fourth threading are of a same handedness.
Clause 10. The delivery pen of any one of clauses 1-9, wherein the inner screw comprises a wide section and a narrow section extending longitudinally from the wide section, wherein the outer housing is an outer screw that concentrically engages at least a portion of the wide section, and wherein the pusher is disposed between the narrow section of the inner screw and the outer screw.
Clause 11. The delivery pen of any one of clauses 1-10, wherein a diameter of the narrow section is less than a diameter of the wide section, such that rotation of the inner screw causes the pusher to extend from the inner screw at a first rate and the inner screw to extend from the outer screw at a second rate, wherein the first rate is greater than the second rate.
Clause 12. The delivery pen of any one of clauses 1-11, further comprising: a torque coupling component configured to at least partially receive the screw mechanism; and a dose knob, wherein the dose knob is configured to rotate the torque coupling component upon depression of the dose knob.
Clause 13. The delivery pen of any one of clauses 1-12, further comprising a dose capture device configured to measure a dose size administered from the delivery pen; wherein the dose capture device is removably coupled to the torque coupling component.
Clause 14. The delivery pen of any one of clauses 1-13, wherein the non-circular shape corresponds to a shape of the plunger so as to allow for unimpeded motion.
Clause 15. The delivery pen of any one of clauses 1-14, wherein the pusher is disposed between the plunger and a distal end of the inner screw, the pusher abutting a proximal side of the plunger and configured to move along a longitudinal axis of the reservoir in response to rotation of the inner screw.
Clause 16. The delivery pen of any one of clauses 1-15, wherein a length of the screw mechanism is dimensioned such that, when the inner screw and the pusher are nested or collapsed, the inner screw and a body of the pusher are contained in the outer housing.
Clause 17. The delivery pen of any one of clauses 1-16, further comprising a torque coupling component configured to at least partially receive the screw mechanism, wherein the torque coupling component comprises one or more inner axial slots at proximal end of the torque coupling component, such that the drive shaft is prevented from disassembling from the torque coupling component when the one or more protruding member translates through the one or more inner axial slots.
Clause 18. The delivery pen of any one of clauses 1-17, wherein the pusher comprises a non-circular distal end configured to engage the plunger, wherein the non-circular distal end corresponds to the non-circular shape of the reservoir to prevent rotation of the pusher in the reservoir.
Clause 19. A delivery pen comprising: a reservoir comprising a non-circular shape; a plunger positioned in the reservoir; and a screw mechanism at least partially inserted within the reservoir, the screw mechanism comprising: a drive shaft comprising one or more protruding member at a proximal end and an elongated member extending longitudinally from the proximal end; an inner screw concentrically engaging the elongated member, the inner screw comprising a wide section and a narrow section extending longitudinally from the wide section; an outer screw concentrically engaging the wide section, the outer screw comprising a circular section rotatably engaging the one or more protruding member, and a non-circular section sized to fit within the reservoir; and a pusher disposed between the narrow section and the outer screw to linearly translate a plunger to dispense a medication from the reservoir.
Clause 20. The delivery pen of clause 19, wherein a diameter of the narrow section is less than a diameter of the wide section, such that rotation of the inner screw causes the pusher to extend from the inner screw at a first rate and the inner screw to extend from the outer screw at a second rate, wherein the first rate is greater than the second rate.
Clause 21. The delivery pen of clause 19 or clause 20, further comprising: a torque coupling component configured to at least partially receive the screw mechanism; and a dose knob, wherein the dose knob is configured to rotate the torque coupling component upon depression of the dose knob.
Clause 22. The delivery pen of any one of clauses 19-21, further comprising a dose capture device configured to measure a dose size administered from the delivery pen; wherein the dose capture device is removably coupled to the torque coupling component.
Clause 23. The delivery pen of any one of clauses 19-22, wherein the non-circular shape corresponds to a shape of the plunger so as to allow for unimpeded motion.
Clause 24. The delivery pen of any one of clauses 19-23, wherein the pusher is disposed between the plunger and a distal end of the inner screw, the pusher abutting a proximal side of the plunger and configured to move along a longitudinal axis of the reservoir in response to rotation of the inner screw.
Clause 25. The delivery pen of any one of clauses 19-24, wherein a length of the screw mechanism is dimensioned such that, when the inner screw and the pusher are nested or collapsed, the inner screw and a body of the pusher are contained in the outer screw.
Clause 26. The delivery pen of any one of clauses 19-25, further comprising a torque coupling component comprising inner axial slots shaped to mate with one or more protruding member of the drive shaft, wherein the torque coupling component comprises one or more slots at proximal end of the torque coupling component, such that the drive shaft is prevented from disassembling from the torque coupling component when the one or more protruding member translates through the inner axial slots.
Clause 27. The delivery pen of any one of clauses 19-26, wherein the pusher comprises a non-circular distal end configured to engage the plunger, wherein the non-circular distal end corresponds to the non-circular shape of the reservoir to prevent rotation of the pusher in the reservoir.
Clause 28. The delivery pen of any one of clauses 19-27, wherein: the wide section of the inner screw comprises a first threading on an outer surface of the wide section; the outer screw comprises a second threading on an inner surface of the outer screw, wherein the first threading and the second threading are engaged such that the inner screw can be longitudinally advanced along the outer screw by rotation of the inner screw; the narrow section of the inner screw comprises a third threading on an outer surface of the narrow section of the inner screw; and the pusher comprises a fourth threading on an inner surface of the pusher, wherein the third threading and the fourth threading are engaged such that the pusher can be longitudinally advanced along the inner screw by rotation of the inner screw.
Clause 29. The delivery pen of any one of clauses 19-28, wherein: the first threading and the third threading are of an opposite handedness; and the second threading and the fourth threading are of an opposite handedness.
Clause 30. A delivery pen comprising: a main body; a cap engaged to the main body; a torque coupling component comprising inner axial slots, wherein the torque coupling component is housed with the main body; a screw mechanism comprising: a drive shaft comprising a proximal end and an elongated member extending distally from the proximal end, wherein the proximal end of the drive shaft comprises one or more protruding member configured to mate with the inner axial slots of the torque coupling component; an inner screw concentrically engaging the elongated member and comprising a first outer threading; an outer housing comprising a circular section and a non-circular section, wherein a proximal end of the circular section comprises an indent configured to engage the one or more protruding member such that the drive shaft can rotate about but cannot longitudinally extend from the indent; and a pusher, wherein an inner surface of the pusher comprises inner threading configured to mate with the first outer threading of the inner screw; a reservoir housed within the cap, the reservoir comprising a non-circular shape, an outlet port at a distal end, and a plunger movable along a longitudinal axis of the reservoir, the plunger configured to engage a distal end of the pusher and to provide a seal with respect to inner walls of the reservoir to prevent fluid provided in a fluid chamber defined on a first side of the plunger from leaking into a portion of the reservoir defined by a second side of the plunger; and a dose knob engaged to the torque coupling component, wherein the dose knob is rotatable with respect to the main body for adjusting a volume of fluid delivery such that rotation of the dose knob in a first direction facilitates axial translation of the drive shaft away from a proximal end of the main body, and rotation of the dose knob in a second direction facilitates axial translation of the drive shaft toward the proximal end of the main body.
Clause 31. The delivery pen of clause 30, wherein: the inner screw comprises a wide section, a narrow section, and an inner surface; the inner surface of the inner screw is keyed to engage a distal end of the drive shaft such that torque applied to the torque coupling component is transferred to the inner screw; the narrow section comprises the first outer threading; and the wide section comprises a second outer threading.
Clause 32. The delivery pen of clause 30 or clause 31, wherein: the outer housing is an outer screw; and the circular section of the outer screw comprises inner threads configured to engage with the second outer threading of the inner screw.
Clause 33. The delivery pen of any one of clauses 30-32, wherein a diameter of the narrow section is less than a diameter of the wide section, such that rotation of the inner screw causes the pusher to extend from the inner screw at a first rate and the inner screw to extend from the outer screw at a second rate, wherein the first rate is greater than the second rate.
Clause 34. The delivery pen of any one of clauses 30-33, wherein: the first outer threading and the second outer threading are of an opposite handedness; and the inner threading of the pusher and the inner threads of the outer screw are of an opposite handedness.
Clause 35. The delivery pen of any one of clauses 30-34, further comprising a dose capture device configured to measure a dose size administered from the delivery pen; wherein the dose capture device is removably coupled to the torque coupling component.
Clause 36. The delivery pen of any one of clauses 30-35, wherein the non-circular shape corresponds to a shape of the plunger so as to allow for unimpeded motion.
Clause 37. The delivery pen of any one of clauses 30-36, wherein the pusher is disposed between the plunger and a distal end of the inner screw, the pusher abutting a proximal side of the plunger and configured to move along the longitudinal axis of the reservoir in response to rotation of the inner screw.
Clause 38. The delivery pen of any one of clauses 30-37, wherein a length of the screw mechanism is dimensioned such that, when the inner screw and the pusher are nested or collapsed, the inner screw and a body of the pusher are contained in the outer housing.
Clause 39. The delivery pen of any one of clauses 30-38, wherein the torque coupling component comprises one or more slots at proximal end of the torque coupling component, such that the drive shaft is prevented from disassembling from the torque coupling component when the one or more protruding member translates through the inner axial slots.
Clause 40. The delivery pen of any one of clauses 30-39, wherein the distal end of the pusher is non-circular, wherein the non-circular distal end of the pusher corresponds to the non-circular shape of the reservoir to prevent rotation of the pusher in the reservoir.
Clause 41. The delivery pen of any one of clauses 30-40, wherein: the drive shaft comprises a second threading on an outer surface of the elongated member; the inner screw comprises a third threading on an inner surface of the inner screw, wherein the second threading and the third threading are engaged such that the inner screw can be longitudinally advanced by rotation of the drive shaft; and the first outer threading of the inner screw and the inner threading of the pusher are engaged such that the pusher can be longitudinally advanced by rotation of the inner screw.
Clause 42. The delivery pen of any one of clauses 30-41, wherein: the first outer threading and the second threading are of a same handedness; and the third threading and the inner threading of the pusher are of a same handedness.
Clause 43. A delivery pen comprising: a non-circular reservoir; and a screw mechanism at least partially inserted within the non-circular reservoir, the screw mechanism comprising: a drive shaft, wherein a proximal end of the drive shaft comprises one or more protruding member; an inner screw comprising a wide section and narrow section, wherein an inner surface of the inner screw is keyed to engage a distal end of the drive shaft such that torque applied to the drive shaft is transferred to the inner screw, the wide section comprises a first outer threading, and the narrow section comprises a second outer threading; an outer screw comprising a circular section and a non-circular section, wherein the circular section comprises inner threads configured to engage with the first outer threading, and wherein a proximal end of the circular section comprises an indent configured to engage the one or more protruding member such that the drive shaft can rotate about but cannot longitudinally extend from the indent; and a pusher distally configured to engage a plunger, wherein an inner surface of the pusher comprises inner threading configured to mate with the second outer threading of the inner screw.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y herein should be interpreted as meaning either “X” or “Y” individually, or both “X and Y” together.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the disclosure. It is intended that the present disclosure be limited only to the extent required by the appended claims and the applicable rules of law.

It is also to be understood that the following claims are to cover all generic and specific features of the disclosure described herein, and all statements of the scope of the disclosure which, as a matter of language, might be said to fall therebetween.

1. A delivery pen comprising:
a reservoir comprising a non-circular shape;
a plunger positioned in the reservoir; and
a screw mechanism at least partially inserted within the reservoir, the screw mechanism comprising:
a drive shaft comprising one or more protruding member at a proximal end and an elongated member extending longitudinally from the proximal end;
an inner screw concentrically engaging the elongated member;
an outer housing comprising a circular section rotatably engaging the one or more protruding member and a non-circular section sized to fit within the reservoir; and
a pusher disposed between the inner screw and the outer housing to linearly translate the plunger so as to dispense a medication from the reservoir.

2. The delivery pen of claim 0, further comprising:
a first protrusion toward a distal end of an outer surface of the elongated member, and
a first interruption toward a proximal end of an inner surface of the inner screw,
wherein the first protrusion is configured to engage the first interruption as the inner screw longitudinally advances along the drive shaft.

3. The delivery pen of claim 0, wherein the first protrusion and the first interruption are positioned such that they are most separated when the drive shaft is fully nested within the inner screw.

4. The delivery pen of claim 0, wherein the first protrusion comprises a thread and the first interruption comprises an inserted plug.

5. The delivery pen of claim 0, further comprising:
a second protrusion toward a distal end of an outer surface of the inner screw, and
a second interruption toward a proximal end of an inner surface of the pusher,
wherein the second protrusion is configured to engage the second interruption as the pusher longitudinally advances along the inner screw.

6. The delivery pen of claim 0, wherein the second protrusion and the second interruption are positioned such that they are most separated when the inner screw is fully nested within the pusher.

7. The delivery pen of claim 0, wherein the second protrusion comprises a thread and the second interruption comprises an inserted plug.

8. The delivery pen of claim 1, wherein:
the drive shaft comprises a first threading at a distal end of an outer surface of the elongated member;
the inner screw comprises a second threading on an inner surface of the inner screw, wherein the first threading and the second threading are engaged such that the inner screw can be longitudinally advanced by rotation of the drive shaft;
the inner screw comprises a third threading at a distal end of an outer surface of the inner screw; and
the pusher comprises a fourth threading on an inner surface of the pusher, wherein the third threading and the fourth threading are engaged such that the pusher can be longitudinally advanced by rotation of the inner screw.

9. The delivery pen of claim 8, wherein:
the first threading and the third threading are of a same handedness; and
the second threading and the fourth threading are of a same handedness.

10. The delivery pen of claim 0, wherein the inner screw comprises a wide section and a narrow section extending longitudinally from the wide section, wherein the outer housing is an outer screw that concentrically engages at least a portion of the wide section, and wherein the pusher is disposed between the narrow section of the inner screw and the outer screw.

11. The delivery pen of claim 10, wherein a diameter of the narrow section is less than a diameter of the wide section, such that rotation of the inner screw causes the pusher to extend from the inner screw at a first rate and the inner screw to extend from the outer screw at a second rate, wherein the first rate is greater than the second rate.

12. The delivery pen of claim 0, further comprising:
a torque coupling component configured to at least partially receive the screw mechanism; and
a dose knob, wherein the dose knob is configured to rotate the torque coupling component upon depression of the dose knob.

13. The delivery pen of claim 12, further comprising a dose capture device configured to measure a dose size administered from the delivery pen; wherein the dose capture device is removably coupled to the torque coupling component.

14. The delivery pen of claim 0, wherein the non-circular shape corresponds to a shape of the plunger so as to allow for unimpeded motion.

15. The delivery pen of claim 0, wherein the pusher is disposed between the plunger and a distal end of the inner screw, the pusher abutting a proximal side of the plunger and configured to move along a longitudinal axis of the reservoir in response to rotation of the inner screw.

16. The delivery pen of claim 0, wherein a length of the screw mechanism is dimensioned such that, when the inner screw and the pusher are nested or collapsed, the inner screw and a body of the pusher are contained in the outer housing.

17. The delivery pen of claim 0, further comprising a torque coupling component configured to at least partially receive the screw mechanism, wherein the torque coupling component comprises one or more inner axial slots at proximal end of the torque coupling component, such that the drive shaft is prevented from disassembling from the torque coupling component when the one or more protruding member translates through the one or more inner axial slots.

18. The delivery pen of claim 1, wherein the pusher comprises a non-circular distal end configured to engage the plunger, wherein the non-circular distal end corresponds to the non-circular shape of the reservoir to prevent rotation of the pusher in the reservoir.

19. A delivery pen comprising:
a reservoir comprising a non-circular shape;
a plunger positioned in the reservoir; and
a screw mechanism at least partially inserted within the reservoir, the screw mechanism comprising:
a drive shaft comprising one or more protruding member at a proximal end and an elongated member extending longitudinally from the proximal end;
an inner screw concentrically engaging the elongated member, the inner screw comprising a wide section and a narrow section extending longitudinally from the wide section;
an outer screw concentrically engaging the wide section, the outer screw comprising a circular section rotatably engaging the one or more protruding member, and a non-circular section sized to fit within the reservoir; and
a pusher disposed between the narrow section and the outer screw to linearly translate a plunger to dispense a medication from the reservoir.

20.-29. (canceled)

30. A delivery pen comprising:
a main body;
a cap engaged to the main body;
a torque coupling component comprising inner axial slots, wherein the torque coupling component is housed with the main body;
a screw mechanism comprising:
a drive shaft comprising a proximal end and an elongated member extending distally from the proximal end, wherein the proximal end of the drive shaft comprises one or more protruding member configured to mate with the inner axial slots of the torque coupling component;
an inner screw concentrically engaging the elongated member and comprising a first outer threading;
an outer housing comprising a circular section and a non-circular section, wherein a proximal end of the circular section comprises an indent configured to engage the one or more protruding member such that the drive shaft can rotate about but cannot longitudinally extend from the indent; and
a pusher, wherein an inner surface of the pusher comprises inner threading configured to mate with the first outer threading of the inner screw;
a reservoir housed within the cap, the reservoir comprising a non-circular shape, an outlet port at a distal end, and a plunger movable along a longitudinal axis of the reservoir, the plunger configured to engage a distal end of the pusher and to provide a seal with respect to inner walls of the reservoir to prevent fluid provided in a fluid chamber defined on a first side of the plunger from leaking into a portion of the reservoir defined by a second side of the plunger; and
a dose knob engaged to the torque coupling component, wherein the dose knob is rotatable with respect to the main body for adjusting a volume of fluid delivery such that rotation of the dose knob in a first direction facilitates axial translation of the drive shaft away from a proximal end of the main body, and rotation of the dose knob in a second direction facilitates axial translation of the drive shaft toward the proximal end of the main body.

31.-42. (canceled)

43. A delivery pen comprising:
a non-circular reservoir; and
a screw mechanism at least partially inserted within the non-circular reservoir, the screw mechanism comprising:
a drive shaft, wherein a proximal end of the drive shaft comprises one or more protruding member;
an inner screw comprising a wide section and narrow section, wherein an inner surface of the inner screw is keyed to engage a distal end of the drive shaft such that torque applied to the drive shaft is transferred to the inner screw, the wide section comprises a first outer threading, and the narrow section comprises a second outer threading;
an outer screw comprising a circular section and a non-circular section, wherein the circular section comprises inner threads configured to engage with the first outer threading, and wherein a proximal end of the circular section comprises an indent configured to engage the one or more protruding member such that the drive shaft can rotate about but cannot longitudinally extend from the indent; and
a pusher distally configured to engage a plunger, wherein an inner surface of the pusher comprises inner threading configured to mate with the second outer threading of the inner screw.
